(19) United States
(12) Patent Application Publication
(10) Pub. No.: US 2024/0010675 A1
(43) Pub. Date: Jan. 11, 2024

(54) PREPARATION OF HIGHLY CONCENTRATED ALLERGENIC INTERMEDIATE

(71) Applicant: Jubilant HollisterStier LLC, Spokane, WA (US)

(72) Inventors: Derek Constable, Spokane, WA (US); Shannon Brown, Spokane, WA (US); Indranil Nandi, Yardley, PA (US)

(73) Assignee: Jubilant HollisterStier LLC, Spokane, WA (US)

(21) Appl. No.: 18/252,354

(22) PCT Filed: Nov. 11, 2021
(86) PCT No.: PCT/US2021/059031

§ 371 Date: May 9, 2023

Related U.S. Application Data
(60) Provisional application No. 63/113,052, filed on Nov. 12, 2020.

(51) Int. Cl. C07K 1/34; A61K 35/36; A61K 9/19

(52) U.S. Cl. CPC C07K 1/34; A61K 35/36; A61K 9/19

(57) ABSTRACT

Disclosed herein is a process for preparing a highly concentrated allergenic intermediate from a dilute allergen extract. This process involves providing a crude extract comprising biological material from an allergenic source, where the biological material comprises a solid phase portion and a liquid phase portion; concentrating the crude extract using pervaporation; adjusting the pH of the concentrated crude extract to 4.0-5.0; treating the concentrated extract with a ketonic solvent to form a precipitate; and recovering the precipitate by filtration to obtain a highly concentrated allergenic intermediate.

### FIELD OF THE INVENTION

The present invention relates to a process for the preparation of a highly concentrated allergen intermediate from a dilute allergen extract.

### BACKGROUND OF THE INVENTION

Allergy is an acquired hypersensitivity disorder of the immune system, and is triggered by exposure to harmless environmental substances known as allergens. The allergic reaction is mediated by the formation of IgE class antibodies directed against allergens of concern. Binding of allergen with these antibodies initiates a cascade of inflammatory responses that manifests the symptoms of allergy that can include asthma, rhinitis, allergic eczema, conjunctivitis, and allergic shock. These responses can be initiated by allergens present in animal dander (e.g., cat, dog, horse, etc.); pollens, such as from flowering plants in the genus Ambrosia in the family Asteraceae; micro fungi, such as from the genus Alternaria; insects, such as mite and cockroach; and foods, such as peanuts and milk.

A pollen allergen extract was first used for the treatment of pollen allergic rhinitis in 1911 (Noon, “Prophylactic Inoculation Against Hayfever,” Lancet 4:1572 (1911)). Since then, allergenic extracts have played an important role in diagnosis and treatment of allergic diseases. These extracts are administered subcutaneously, orally, or as skin patches.

The preparation of allergenic intermediates involving efficient recovery of allergenic extracts from allergenic source materials typically revolves around 3 factors: (i) source material characteristics, (ii) processing of the material to obtain an allergen extract (or allergenic intermediate), and (iii) extract storage. The source material characterization depends upon the source from which the allergen is to be extracted. Processing involves extraction of allergen from source material and bringing the allergenic intermediate into a state to be useful for diagnosis or treatment. Once prepared, the correct storage conditions play a role in keeping the extract stable and sterile to meet regulatory standards for distribution and use.

Of all that is involved in obtaining an allergenic intermediate, a significant challenge lies at the processing stage. The first step in processing typically involves contacting a source material, i.e., the solid allergenic biological raw material (e.g., animal dander) with a liquid extracting solution. Once contacted with a liquid extracting solution to form a mixture of source material and extracting solution, the mixture is stirred, milled, or sonicated to release allergenic protein from the solid source material. The allergenic protein is then subjected to separation to separate solids from liquids. Since the amount of allergenic protein is proportionally small compared to the bulk of source solid material from which it is extracted, an efficient process is required to extract and recover the desired material and to minimize loss in waste. At the same time, the process needs to be convenient as well as time and energy efficient. A typical process of filtration for separation of solids from liquids employs centrifugation and dead end filtration, individually or in sequence, to carry out separation, clarification, and filtration. Dead end filtration involves use of membranes of progressively smaller pore size to yield a particulate free solution. Such a process has limitations as it involves loss of products at each stage of separation and clarification whenever a smaller pore size filtration step takes place to remove particulates from solution. Thus, to get a desired solution, the process requires carrying out multiple clarifications, and with each clarification, there is loss of yield, which multiplies the loss in terms of time, energy, and process capability to produce desired results. Another operational disadvantage associated with a typical process is blockage of dead end filters which is due to a high solid to liquid ratio and again leads to loss of yield and operational difficulties.

Allergenic extracts are water soluble biological components, often comprised of a complex mixture of proteins, carbohydrates, enzymes, dyes, etc. One of the major risks involved with use of allergenic extracts is that their administration to a sensitized patient can cause severe allergic reactions or anaphylaxis. Thus, preparation and maintenance of a safe and effective allergenic extract using a robust method is important to ensure its purity, potency, safety, stability, and sterility during preparation, handling, storage, and distribution.

Active allergenic extracts typically include protein allergens having a molecular weight of about 10-100 kDa, whereas molecules with a molecular weight lower than 10 kDa may bring out additional toxic or irritant side effects in patients. Accordingly, efficient removal of low molecular weight molecules is a desirable objective for allergenic extract preparation.

It is desirable for allergenic extracts to be produced as highly concentrated biological solutions. However, there are several factors that limit how concentrated an allergen immunotherapy extract can be, including the tendency of highly concentrated antigen solutions to develop precipitates. This is an unpredictable and poorly understood phenomenon. Although there is no evidence that such precipitates adversely affect allergenic extracts, the U.S. Food and Drug Administration, through implementation of the U.S. Pharmacopeia general chapter <1> Injections, does not permit a manufacturer to ship an extract that has a precipitate.

Precipitation arises due to aggregation of soluble components to form insoluble particulates, reactions, or by a combination of components to form insoluble salts, rendering an allergenic extract unsuitable for immunotherapy and other purposes despite having been prepared by a lengthy and tedious process. Consequently, special emphasis must be directed to the manufacturing process to obtain yields more consistent in lot-to-lot composition and having the requisite stability to meet regulatory requirements and customer expectations. U.S. patent application Ser. No. 16/678,432, filed on Nov. 8, 2019, describes processes for the preparation of an allergenic extract, which involve efficient recovery of allergenic extract from allergenic source material. The allergenic extracts obtained are stable, and upon reconstitution have substantially reduced amounts of, or are devoid of, any precipitation due to aggregation, chemical reaction, salt formation, etc., compared to allergenic extracts prepared from other processes.

U.S. Pat. No. 2,316,311 discloses a process for the preparation of allergenic extracts. According to this process, raw materials are extracted into an aqueous extracting fluid and then purified or concentrated by subjecting the extract to fractional precipitation by the addition of a suitable ether, ketone, or mono-hydroxy alcohol. The most preferred solvent used was acetone and subsequent treatment with a salt, such as ammonium sulfate, sodium sulfate, or zinc sulfate. This process suffers from inefficient removal of low molecular weight molecules, and so it is no longer used.

U.S. Pat. Nos. 3,148,121 and 3,148,122 disclose a process for preparing allergenic extracts, which involves treating whole allergenic substances with an aqueous heterocyclic amine extracting fluid, separating the liquid phase containing the active principles, and discarding the residue. To separate the active substances from the heterocyclic amine, water and an alum solution are added in order to precipitate the allergenic components. Since the most commonly used heterocyclic amine was pyridine, the process is commonly called the pyridine extracted alum precipitated (“PEAP”) process. Over the years, several limitations appeared with allergenic extracts prepared by the PEAP process. For example, pyridine used during the extraction alters the natural structure of the extract, rendering it largely inactive and not suitable for skin testing purposes. Further, the process is cumbersome with yields in the range of about 50% based on the Protein Nitrogen Unit (PNU) content of the original extract. Thus, this process is not suitable for preparing allergenic extracts on a commercial scale with acceptable yields.

U.S. Pat. No. 3,591,677 discloses a process for preparing an extract useful for hyposensitization therapy involving precipitating the active principles using an aluminum compound such as potassium aluminum sulfate. This process initially involves the treatment of aqueous extracted fluid with a heterocyclic tertiary amine, such as pyridine, picoline, lutidine, etc. Since the process also involves use of heterocyclic amines, which need high temperature treatment for their removal from extracted allergenic substances, denaturing of allergens can occur. Therefore, this process has limitations. Further, the extracts obtained from this process are not suitable for diagnostic purposes.

U.S. Pat. No. 3,953,588 discloses a process involving pre-extraction, where the allergen-containing material is treated with a lipophilic solvent, which is a cyclic ether selected from the group of tetrahydrofuran, tetrahydropyran, and dioxane before aqueous extraction of allergen material. This pre-treatment not only removes any lipophilic portion present in allergen-containing material, but also takes along with it some portion of allergen contacting material. Thus, this process involves not only the use of ethers having mutagenic effects, but also leads to yield loss. Accordingly, this process has limitations based on safety concerns and low yield.

U.S. Pat. No. 4,234,569 discloses a method for production of allergen extracts, which involves the treatment of allergen extract with formaldehyde or lower saturated aliphatic aldehyde which modifies the allergen. Thus, this process does not provide allergens in their natural form, but rather they are provided in modified form, which is not effective for diagnosis and may reduce efficacy in immunotherapy.

U.S. Pat. No. 5,770,698 discloses a method for the preparation of an allergenic extract, which involves preparing an aqueous extract containing allergenically active proteins to which undesirable non-allergenic compounds (e.g., low molecular weight compounds) are adhered. This process involves disrupting electrostatic, hydrophobic, or other physical forces. Under such conditions, the non-allergenic compounds disadhere from allergenically active proteins. The disruption is brought about by treatment with an acid having a pH value of less than 3, or by electric currents in the form of electrophoresis. Thus, the process adds operations, which makes the extraction process lengthier, and acid treatment and electric treatment may alter the allergen extract structure making it less effective. Thus, this process is not ideally suited for allergen extract preparation.

JP Patent No. 3,302,418 discloses a process for the preparation of Dermatophagoides farinae mite allergen extract. This process involves treating the mite bodies with saturated sodium chloride, allowing the treated mite bodies to stand, and then subjecting the resulting solid-liquid mixture to centrifugation. Mite bodies are then separated by filtration and the filtrate is subjected to ultrafiltration to remove low molecular weight components. However, this process suffers from an inefficient recovery of extract from mite bodies. Specifically, floating mite bodies have to be removed by filtration, which also filters out allergen components, leading to a loss of yield of the allergen extract. Consequently, this process is not viable for large scale applications.

U.S. Pat. No. 7,887,821 discloses a process for the preparation of an allergenic extract, specifically a pollen allergen, by extraction, dead end filtration, first concentration, diafiltration, conductivity test, second concentration, dry weight adjustment, and clarification by filtration prior to storage. The disclosed process during extraction involves mixing a source material with an extraction buffer in a ratio of 1:10, agitation at 8° C. for 2 hours, and adjusting the pH during extraction from 5 to 9. After extraction, further steps are performed, including diafiltration until a desired conductivity test result is obtained. The process aims to remove low molecular weight components that are potentially toxic from allergen extracts, but is tedious in terms of pH adjustment and carrying out conductivity tests as an end point for diafiltration, which adds steps and requires careful monitoring of the process. This increases the time cycle and cost of the process, making it less than suitable for commercial scale.

U.S. Patent Application Publication No. 2013/195888 discloses the utilization of widely used ultrafiltration and diafiltration processes for extraction of antibody and antigen binding protein products. The resultant extract is further treated with buffer or surfactant to avoid aggregation or precipitation in final preparation. Thus, the disclosed process has a shortcoming in terms of requiring the addition of buffers and/or surfactant additives to make the product stable, and which may alter the composition if applied to allergenic extracts.

Accordingly, it is understood that known allergen extraction processes suffer from many disadvantages. These include the use of heterocyclic amines like pyridine for precipitation, which may alter the allergens; employing sophisticated techniques like electrophoresis, which add to cost or require the performance of unnecessary pH adjustments/conductivity tests; or utilizing methods that reduce efficiency of the allergenic extraction process through lower yields, including the use of additives to stabilize the finished product.

Allergenic extracts used for diagnosis are typically tested in the skin of a patient by skin testing (e.g., scratch, puncture, intradermal, etc.) to deliver a minute volume (ranging from nanoliters to microliters) of extract into the skin. To achieve a useful diagnostic capability, an extract solution must contain a high level (i.e., highly concentrated amount) of the allergen intermediate. Certain extracts, such as allergens from animal hair and dander, are difficult to achieve in high concentration and are therefore often more dilute. Yet, dilute extracts have limited effectiveness in diagnostic settings. Highly concentrated extracts prepared using an allergen intermediate are more useful for achieving an acceptable diagnostic level.

An allergenic intermediate for clinical use is typically subjected to reformulation in a suitable medium and mixed with other allergen intermediates to form a single immunotherapy treatment solution. A mixed allergen formulation must contain each of the individual allergens at a concentration such that a therapeutic level of each can be injected into a patient (e.g., via the patient's skin) in the smallest volume possible. To increase the ability of a single prescription to include multiple allergen intermediates of varied sources, it is highly desirable for the allergenic intermediate being used to be highly concentrated. A highly concentrated allergenic intermediate enables the co-formulation of multiple allergenic intermediates, but is often difficult to achieve, especially for certain types of allergens, including allergens from animal hair and dander.

Further, such formulations containing multiple allergenic intermediates usually contain about 50% glycerin. Injections having such a high amount of glycerin may be painful to a patient when a large volume needs to be injected. Therefore, it would be desirable to prepare highly concentrated allergenic intermediates from dilute allergen extracts, including combinations of multiple allergenic intermediates at dilutions that enable the reduction of the effective volume of glycerin extract required.

The present disclosure is directed to overcoming deficiencies in the art.

### SUMMARY OF THE INVENTION

One aspect of the invention relates to a process for preparing a highly concentrated allergenic intermediate from a dilute allergen extract. This process involves providing a crude extract comprising biological material from an allergenic source, where the biological material comprises a solid phase portion and a liquid phase portion; concentrating the crude extract using pervaporation; adjusting the pH of the concentrated crude extract to 4.0-5.0; treating the concentrated extract with a ketonic solvent to form a precipitate; and recovering the precipitate by filtration to obtain a highly concentrated allergenic intermediate. In one embodiment, the intermediate can then be reconstituted to form a highly concentrated extract.

For various allergen related treatments, allergenic intermediates are commonly obtained by the combination of solid allergenic biological source material (e.g., animal epithelia, hair, or dander) with a liquid solution. Animal epithelia, hair, and/or dander source materials are typically very low in allergen content. For example, cat and dog epithelia and hair dander source material is much lower in allergen content compared to pollen and mold source material. Traditional processes used for the preparation of allergen extracts of these low allergen content materials have a physical limitation based on the volume of liquid that is retained by the source material. Depending on the desired concentration, the recovered volume can be very small or may not be physically achievable. Thus, understanding the need of highly concentrated allergen intermediates, including from dilute allergen extracts, the inventors of the present invention provide an efficient and simple process that achieves these requirements.

The process of the present invention enables the production of highly concentrated allergenic intermediates capable of delivering minute volumes (ranging from nanoliters to microliters) of highly concentrated extract into e.g., a patient's skin to achieve a useful diagnostic capability. The process of the present invention can produce allergenic intermediates at a concentration high enough to enable co-formulation of multiple allergenic intermediates, even using allergens from animal hair and dander. In addition, the process of the present invention enables formulations containing multiple allergenic intermediates to be effectively reformulated in, e.g., 50% glycerol medium and mixed with other allergen intermediates to form a single, effective, immunotherapy treatment solution that can deliver an effective dose in a substantially reduced volume causing less pain to the patient. The present invention overcomes the drawback of prior processes, which diminished the returns of allergen extract where the source material has a low content of allergens and only allowing dilute solutions to be produced. The inventive process allows the production of highly concentrated solutions in high yield that are not achievable by processes that solely depend upon liquid to solid extraction and therefore allows the required quantity to be injected in small volume.

Allergen intermediates obtained by the process disclosed herein are capable of being formulated into a final solution having an allergen concentration in the range of at least 10,000 to 90,000 BAU/mL.

### BRIEF DESCRIPTION OF THE FIGURES

FIG. 1 is a graphic depiction of the Sensitivity, Specificity, PPV and NPV by Responder Groups and Titrated SPT using Cat Allergens (FAS Population) which were calculated using the data generated in the clinical study described in Example 2.

FIG. 2 is a graphic depiction of the summary of maximum mean wheal diameter by cat hair allergens and responder groups (FAS Population) which were calculated using the data generated in the clinical study described in Example 2.

FIG. 3 is a graphic depiction of the summary of maximum mean erythema diameter by cat hair allergens and responder groups (FAS Population) which were calculated using the data generated in the clinical study described in Example 2.

### DETAILED DESCRIPTION

One aspect of the invention relates to a process for preparing a highly concentrated allergenic intermediate from a dilute allergen extract. This process involves providing a crude extract comprising biological material from an allergenic source, where the biological material comprises a solid phase portion and a liquid phase portion; concentrating the crude extract using pervaporation; adjusting the pH of the concentrated crude extract to 4.0-5.0; treating the concentrated extract with a ketonic solvent to form a precipitate; and recovering the precipitate by filtration to obtain a highly concentrated allergenic intermediate. In one embodiment, the intermediate can then be reconstituted to form a highly concentrated extract.

In one embodiment, providing a crude extract according to the process described herein involves providing a crude extract; contacting an allergenic source material with an extraction fluid; extracting biological material from the allergenic source into the extraction fluid, wherein said extracting is carried out with no adjustment to pH; and separating the biological material into a solid phase portion and a liquid phase portion to obtain a crude extract.

In one embodiment, an allergenic source material is extracted in a larger volume of liquid or repeated volumes of liquid to maximize recovery of the allergen in the extract. This approach is necessary due to the relatively low concentration of allergens in the allergenic source material. In effect, the process is carried out to produce a large volume of diluted extract that is then concentrated by pervaporation (to reduce water content), followed by a pH adjustment and then precipitation of the allergenic fraction by addition of ketonic solvent. Precipitation with ketonic solvent may be repeated. From this is recovered a solid quantity of allergen that is devoid of water content and is high in allergen content.

The allergenic extract, also referred to as an allergenic intermediate, is an extract of a biological allergen source material containing any naturally occurring protein allergen that induces allergic (i.e., IgE mediated) reactions upon repeated exposure to an individual. Examples of naturally occurring allergens include animal hair and dandruff allergens (from e.g., dog, cat, horse, rabbit, rat, mouse, etc.), pollen allergens (e.g., tree, herb, weed, and grass pollen allergens), insect allergens (e.g., inhalant, saliva, and venom allergens, such as from mite, cockroach, midge, or hymenoptera venom), and food allergens.

In one embodiment, the source material is from a mammal, such as cat, dog, horse, rabbit, etc. The mammalian source material may be from hair, dander, saliva, or other source of allergenic proteins.

In another embodiment, the source material is pollen, e.g., a pollen allergen from trees, grasses, and herbs are such originating from the taxonomic orders of Fagales, Oleales, Pinales and platanaceae including, among others, birch (Betula), alder (Alnus), hazel (Corylus), hornbeam (Carpinus) and olive (Olea), cedar (Cryptomeria and Juniperus), Plane tree (Platanus), the order of Poales including, among others, grasses of the genera Lolium, Phleum, Poa, Cynodon, Dactylis, Holcus, Phalaris, Secale, and Sorghum, the orders of Asterales and Urticales including, among others, herbs of the genera Ambrosia, Artemisia, and Parietaria.

In another embodiment, the source material is from house dust mites of the genus Dermatophagoides and Euroglyphus, storage mites (e.g., Lepidoglyphys, Glycyphagus, and Tyrophagus), or other insect-like arthropods.

In another embodiment, the source material is an insect source material, e.g., from cockroaches, midges, and fleas (e.g., Blatella, Periplaneta, Chironomus, and Ctenocepphalides). Other important source materials are venom allergens, including such originating from stinging or biting insects such as those from the taxonomic order of Hymenoptera including bees (superfamily Apidae), wasps (superfamily Vespidea), and ants (superfamily Formicoidae).

In another embodiment, the source material is from common inhalation allergen sources, e.g., from fungi and molds including, for example, those originating from the genera Alternaria and Cladosporium.

Several allergens are known and may be extracted in the processes described herein. Such allergens may include, for example and without limitation, Bet v 1, Aln g 1, Cor a 1, Car b 1, Que a 1, Cry j 1, Cry j 2, Cup a 1, Cup s 1, Jun al, Jun a 2, Jun a 3, Ole e 1, Lig v 1, Pla 1 1, Pla a 2, Amb a 1, Amb a 2, Amb t 5, Art v 1, Art v 2 Par j 1, Par j 2, Par j 3, Sal k 1, Ave e 1, Cyn d 1, Cyn d 7, Dac g 1, Fes p 1, Hol 1 1, Lol p 1 and 5, Pha a 1, Pas n 1, Phl p 1, Phl p 5, Phl p 6, Poa p 1, Poa p 5, Sec c 1, Sec c 5, Sor h 1, Der f 1, Der f 2, Der p 1, Der p 2, Der p 7, Der m 1, Eur m 2, Gly d 1, Lep d 2, Blot 1, Tyr p 2, Bla g 1, Bla g 2, Per a 1, Fel d 1, Can f 1, Can f 2, Bos d 2, Equ c 1, Equ c 2, Equ c 3, Mus m 1, Rat n 1, Apis m 1, Api m 2, Ves v 1, Ves v 2, Ves v 5, Dol m 1, Dil m 2, Dol m 5, Pol a 1, Pol a 2, Pol a 5, Sol i 1, Sol i 2, Sol i 3 and Sol i 4, Alt a 1, Cla h 1, Asp f 1, Bos d 4, Mal d 1, Gly m 1, Gly m 2, Gly m 3, Ara h 1, Ara h 2, Ara h 3, Ara h 4, and Ara h 5. Other allergens also exist and will be discovered, and may be extracted using the processes described herein.

In one embodiment, the allergenic intermediate obtained via the process described herein comprises Can f 1 and/or Fel d 1 from animal epithelia.

In one embodiment, the allergenic intermediate obtained via the process described herein comprises Der p 1 and/or Der f 1 from house dust mite.

In one embodiment, the allergenic intermediate obtained via the process described herein comprises Phl p 5 and/or Phl p 1 from grass pollen.

In one embodiment, the allergenic intermediate obtained via the process described herein comprises Alt a 1 and/or Asp f 1 from mold.

In one embodiment, the allergen obtained from the process described herein comprises any one or more of a cat allergen, a dog allergen, a tree pollen allergen, a grass pollen allergen, a house dust mite allergen, a storage mite allergen, a weed allergen, or a mold allergen.

In one embodiment, the allergen obtained from the process described herein comprises a cat allergen or a dog allergen.

According to one embodiment, the extraction fluid used in the process of the present invention may be, for example and without limitation, an aqueous solution of glycerol, sodium chloride, and sodium bicarbonate; or an aqueous solution of sodium chloride, sodium bicarbonate, and a ketonic solvent. In one embodiment, the phenolic compound is selected from a phenol, catechol, and the like. Other extraction fluids known by those of ordinary skill in the art may also be used. Suitable extraction fluids include those capable of contacting an allergenic source material, typically in solid form, and extracting biological material (e.g., allergenic proteins) from the allergenic source into the extraction fluid. Such extraction creates a mixture containing a solid phase and a liquid phase portion, which is referred to as a crude extract.

In one embodiment, the allergenic source material is contacted with the extraction fluid at a concentration ratio of 1:4 to 1:8. In one embodiment, the allergenic source is contacted with the extraction fluid at a concentration ratio of about 1:4, 1:5, 1:6, 1:7, 1:8, or about 1:4.0, 1:4.1, 1:4.2; 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, or 1:8.0, or other decimal variation thereof or range of ratios thereof. As used herein, the term “about” means ±1%, 2%, 3%, 4%, 5%, 6%, 7%, 8%, 9%, or 10% of the stated value.

In the methods described herein, the extraction process may be carried out for about 2 days to about 6 days, although other time frames may also be useful, including for several hours up to at least a week or longer. In one embodiment, the extraction process is carried out for about 6 hours, 12 hours, 18 hours, 24 hours, 30 hours, 36 hours, 42 hours, 48 hours, 2.5 days, 3 days, 3.5 days, 4 days, 4.5 days, 5 days, 5.5 days, 6 days, 6.5 days, 7 days, or 7.5 days, or any fraction or range of time thereof.

In the process of the present invention, the extraction process may be carried out at a temperature of about −5° C. to about 30° C., or at a temperature of about −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., or any fraction thereof or within any range thereof.

According to one embodiment, the extracted source material is liquid phase separated from the solid source material residue, particularly large particles, to recover a crude extract. Such separation may be carried out using techniques known in the art including, without limitation, dead end filtration, centrifugation, and the like, typically at ambient temperature, although other conditions may also be used. According to one embodiment, separation of a solid phase portion from a liquid phase portion is carried out by centrifugation or dead end filtration, or by centrifugation and then dead end filtration. In one embodiment, separating is carried out using depth filtration, centrifugation, or a combination thereof.

In one embodiment, once the biological material has been separated into a solid phase portion and a liquid phase portion to obtain/provide a crude extract according to the process of the present invention, the crude extract is concentrated using pervaporation. Pervaporation is a separation technology where a liquid mixture, referred to as the “feed” is placed into contact with a first side of a membrane and the permeate is removed as a low-pressure vapor from the other side of the membrane.

In one embodiment, pervaporation is carried out using dialysis at room temperature. Pervaporation may be carried out or continued until a concentration is achieved to about 20% of its original volume, or about 19%, 18%, 17%, 16%, 15%, 14%, 13%, 12%, 11%, 10%, 9%, 8%, 7%, 6%, 5%, or lower, or any fraction or range thereof of the original volume.

According to one embodiment, the pH of the pervaporated extract is adjusted to about 4.0 to about 6.0, or about 4.0 to about 5.0, or to about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, or any fraction thereof or range therein.

In one embodiment, concentrating using pervaporation is carried out in the absence of a conductivity test.

In carrying out the process of the present invention, the concentrated extract is then treated with a ketonic solvent. In one embodiment, the concentrated extract is mixed with 2-6 volumes of ketonic solvent, or about 2, 3, 4, 5, 6 (or any fraction thereof or range within) volumes of ketone solvent. In one embodiment, the ketonic solvent is selected from acetone, methyl ethyl ketone, and the like. In one embodiment, addition with ketonic solvent is carried out with a chilled ketonic solvent. In one embodiment, after the addition of ketonic solvent, the resulting solution is left to stand for about 1-48 hours, or about 1, 2, 3, 4, 5, 6, 7, 8, 9 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, or any fraction thereof or range therein, at room temperature (or other suitable temperature) to allow precipitation, and then the solvent is decanted off. In one embodiment, the precipitation process is repeated. For example, the precipitation process may be repeated 2-4 times, or more, to obtain the precipitate, which functions to increase concentration of the allergenic intermediate.

In carrying out the process of the present invention, the room temperature used is temperature between 20° C.-25° C.

In carrying out the process of the present invention, the precipitate is subjected to filtration to obtain a precipitated extract as retentate. In other words, the process involves recovering the precipitate by filtration.

In one embodiment, the allergenic intermediate of the present invention is used for reconstitution in various diluents at predetermined concentrations for one or more uses. Such uses may include, for example and without limitation, skin prick testing, immunotherapy vaccines, incorporation into solid dosage forms for oral treatment, solid phase adsorption to membranes, etc., including for use in in vitro diagnostic devices. The lyophilized or vacuum dried extract according to the present invention may be used for reconstitution, as per the desired strength of the final solution. For example, and without limitation, in the case of cat allergen extracts, the concentration of the final solution can be in the range of at least about 10,000 to at least about 90,000 BAU/mL, or even higher (e.g., about 95,000 BAU/mL; 100,000 BAU/mL; 105,000 BAU/mL; 110,000 BAU/mL; 115,000 BAU/mL; or 120,000 BAU/mL).

The processes described herein provide a means of concentrating a protein (i.e., an allergenic agent) to very high levels in solution from an otherwise dilute allergenic extract. By “dilute allergen extract,” it is meant that an allergen extract, provided by any means known in the art, has a relatively low concentration of allergen such that the extract is limited in its ability to function in a desirable way. In contrast to dilute allergen extracts, highly concentrated allergen extracts obtained by methods disclosed herein can be more effectively reformulated into, e.g., a suitable medium along with other allergen intermediates to form a single allergy treatment solution. The concentration of the protein in the aqueous formulation obtained using the methods described herein can be any amount in accordance with the desired concentration. Allergen extracts derived from cat and pollen are often characterized by units of measure referred to as “BAU” or “bioequivalent allergy units.” For other allergens such as from dog, the terminology is w/v, such as 1:20 w/v, which stands for 1 g source material per 20 mL of fluid. The relationship between BAU and 1:20 w/v depends upon the extract. In any event, there is a defined amount of extract contained within the concentrate. In one embodiment, allergenic extracts obtained by the processes described herein may be obtained at potency levels of at least about 10,000 BAU/mL; 20,000 BAU/mL; 30,000 BAU/mL; 40,000 BAU/mL; 50,000 BAU/mL; 60,000 BAU/mL; 70,000 BAU/mL; 80,000 BAU/mL; 90,000 BAU/mL, 95,000 BAU/mL, 100,000 BAU/mL, 105,000 BAU/mL, 110,000 BAU/mL, 115,000 BAU/mL, 120,000 BAU/mL, or any level (including fraction thereof) or range among 10,000 BAU/mL to 120,000 BAU/mL. In one particular embodiment, allergenic extracts obtained by the processes described herein are obtained at a potency level of at least 30,000 BAU/mL. In one embodiment, allergenic extracts obtained by the processes described herein are obtained at conventional yields, such as 1:10 w/v. However, application of the processes described herein allow significantly more concentrated allergenic extracts to be obtained, including concentrations that are a 10-100-fold increase (or any number or range therein) in concentration over conventional yields, such as 1:10 w/v.

In one embodiment, once the precipitate is recovered by filtration, it is subjected to lyophilization (e.g., distributed into lyophilization trays and then freeze dried) or vacuum drying to obtain a highly concentrated allergenic intermediate.

As known to the skilled person in the art, allergy and allergic symptoms can be assessed using tests such as a skin prick test, a nasal provocation test, a bronchial provocation test and/or a conjunctival provocation test. A preferred skin prick test in accordance with the present invention is the one described in example 2.

In another preferred embodiment, reduction in the level or severity of the allergic response or reduction of allergic symptom of said human, is monitored by a skin prick test, nasal provocation test or conjunctival provocation test, preferably by a skin prick test as preferably exemplified in the example section of the present invention.

The “Skin Prick Test” (SPT) is a reliable method to diagnose IgE-mediated allergic disease in patients with suspected food and drug allergy, allergy due to animal dander (e.g., cat, dog, horse, etc.); pollens, such as from flowering plants in the genus Ambrosia in the family Asteraceae; micro fungi, such as from the genus Alternaria; insects, such as mite and cockroach; and foods, such as peanuts and milk etc. It provides evidence for sensitization and can help to confirm the diagnosis of a suspected allergy. In the prick test, a small amount of purified allergen is generally applied to the skin, and a device is used to prick the skin surface at the application site. When relevant allergens are introduced into the skin, specific IgE bound to the surface receptors on mast cells are cross-linked, mast cells degranulate, and histamine and other mediators are released. This produces a wheal and flare response, which can be quantitated. SPT results correlate with those of nasal challenge which may also be used as a surrogate to test clinically relevant sensitization.

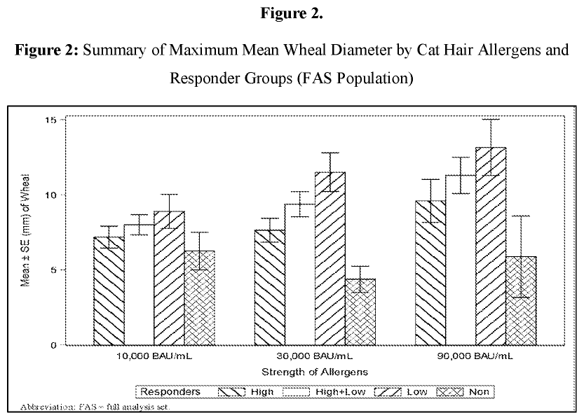

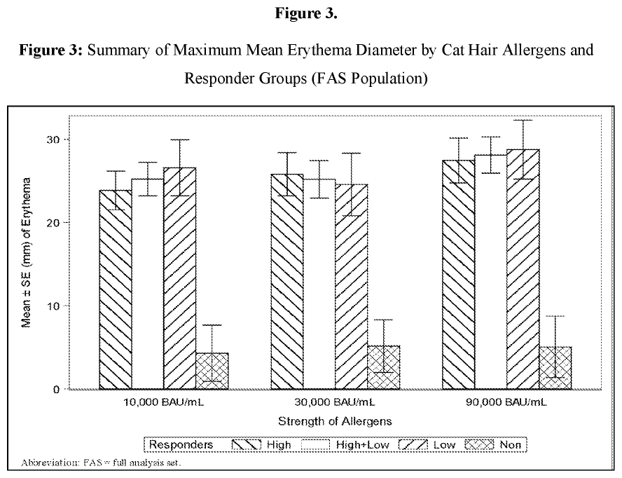

Reactions to SPT typically will manifest as a “skin wheal” or “skin erythema” which is a visually apparent spot (red and/or puffy appearance) of inflammation on the skin. The diameter of the skin wheal and/or erythema bears some relationship to the severity of the patient's allergy. Typically, by current convention, a skin wheal with a diameter of 3 mm more than the negative control, or greater, is indicative of a possible allergic reaction, while skin wheal diameter of less than 3 mm greater than the negative control are not considered reliable indicators of allergy. In one embodiment, the method is associated with a reduction in wheal and/or erythema diameter in an allergen skin test. In one embodiment, the allergen skin test is a skin prick test (SPT), wherein there is a significant reduction in wheal and/or erythema diameter in a patient treated with the allergenic extract as per the present invention compared to the wheal and/or erythema diameter observed prior to treatment of the patient. A preferred skin prick test in accordance with the present invention is the one described in FIGS. 2 and 3.

The term “sensitivity” refers to probability that a SPT using the present invention will be positive for a patient with the disease. Sensitivity essentially is a measure of how well a diagnostic method using the present invention correctly identifies those with disease or allergy. Sensitivity is calculated as the number of true positive results divided by the sum of the true positives and false negatives. The values can be selected such that the sensitivity is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 95%, 96%, 97%, 98%, 99% or 100%. Preferably, the sensitivity is about 80% to about 100%. Preferably, the sensitivity is about 100%. Preferably, the sensitivity is about 90%. More preferably, the sensitivity is about 90.91% or about 95.45%.

The term “specificity” refers to probability that a SPT using the present invention will be negative for a patient that does not have the disease. Specificity essentially is a measure of how well a diagnostic method using the present invention recognizes those that do not have the disease. Specificity is calculated as number of true negative results divided by the sum of the true negatives and false positives. The values can be selected such that the sensitivity is at least about 50%, and can be, for example, at least about 60%, 65%, 70%, 75%, 76%, 77%, 78%, 79%, 80%, 81%, 82%, 83%, 84%, 85%, 86%, 87%, 88%, 89%, 90%, 95%, 96%, 97%, 98%, 99% or 100%. Preferably, the specificity is about 60% to about 100%. Preferably, the specificity is about 100%.

The term “negative predictive value” or “NPV” refers to the probability that an individual truly does not have the disease (i.e. allergy) if the diagnostic test using the present invention is negative. Negative predictive value can be calculated as the number of true negatives divided by the sum of the true negatives and false negatives. Negative predictive value is determined by the characteristics of the diagnostic method as well as the prevalence of the disease or allergy in the population analyzed. The values can be selected such that the negative predictive value in a population having a disease or allergy prevalence is in the range of about 70% to 100% and can be, for example, at least about 70%, 75%, 80%, 85%, 90%, 91%, 92%, 93%, 94%, 95%, 96%, 97%, 98%, 99% or 100%. Preferably, NPV is at least 75%. Preferably, NPV is about 75% to about 100%. Preferably, NPV is about 100%.

The term “positive predictive value” or “PPV” refers to the probability that an individual truly does have the disease (i.e. allergy) if the diagnostic testing using the present invention is positive. Positive predictive value can be calculated as the number of true positives divided by the sum of the true positives and false positives. Positive predictive value is determined by the characteristics of the diagnostic method as well as the prevalence of the disease in the population analyzed. The values can be selected such that the positive predictive value in a population having a disease prevalence is in the range of about 80% to 100% and can be, for example, at least about 70%, 80%, 85%, 90%, 91%, 92%, 93%, 94%, 95%, 96%, 97%, 98%, 99% or 100%. Preferably, PPV is at least 80%. Preferably, PPV is about 80% to about 100%. Preferably, PPV is about 100%.

In one embodiment, the present invention provides a method of diagnosing cat allergy in an individual.

In another embodiment, the present invention provides a method of diagnosing cat allergy in an individual using skin prick method.

In another embodiment, the present invention provides a method of diagnosing cat allergy in an individual using skin prick method, wherein clinical parameters are selected from the group consisting of sensitivity, specificity, negative predictive value, and positive predictive value.

In another embodiment, the method of diagnosing cat allergy, wherein the sensitivity of said method for diagnosing cat allergy is at least 90%. In another embodiment, the sensitivity is 100%.

In another embodiment, the method of diagnosing cat allergy, wherein the specificity of said method for diagnosing cat allergy is at least 60%. In another embodiment, the specificity of said method for diagnosing cat allergy is 100%.

In another embodiment, the method of diagnosing cat allergy, wherein the negative predictive value in a population having a cat allergy prevalence of about 75% to about 100%. In a further embodiment, negative predictive value is at least about 75%. In a further embodiment, negative predictive value is at least about 85%. In a further embodiment, negative predictive value is at least about 88%. In a further embodiment, negative predictive value is about 100%.

In another embodiment, the method of diagnosing cat allergy, wherein the positive predictive value in a population having a cat allergy prevalence of about 80% to about 100%. In a further embodiment, positive predictive value is at least about 80%. In a further embodiment, positive predictive value is at least about 83%. In a further embodiment, positive predictive value is at least about 84%. In a further embodiment, positive predictive value is at least about 91%. In a further embodiment, positive predictive value is about 100%.

In another embodiment, there is provided a method of diagnosing cat allergy in an individual by skin prick method, wherein clinical parameters are selected from the group consisting of sensitivity, specificity, negative predictive value, and positive predictive value and wherein the sensitivity of said method is at least 90%, specificity is at least 60%, negative predictive value in a population having a cat allergy prevalence of about 75% to about 90% and positive predictive value in a population having a cat allergy prevalence of about 80% to about 95%.

In another embodiment, there is provided a method of diagnosing cat allergy in an individual by skin prick method, wherein clinical parameters are selected from the group consisting of sensitivity, specificity, negative predictive value, and positive predictive value and wherein the sensitivity of said method is at least 90%, specificity is at least 60%, negative predictive value in a population having a cat allergy prevalence of at least 75% and positive predictive value in a population having a cat allergy prevalence of at least 80%.

In another embodiment, there is provided a kit for compounding allergenic intermediate comprising:
a) a first container comprising a lyophilized allergenic intermediate,
b) a second container comprising a liquid diluent, and
c) optionally instructions for preparation and use,
   wherein the content of first and second containers can be combined to achieve 10,000, or 90,000 BAU/mL concentration of allergenic extract. In certain non-limiting embodiments, there is provided a method of diagnosing cat allergy in an individual by skin prick method using the kit, wherein clinical parameters are selected from the group consisting of sensitivity, specificity, negative predictive value, and positive predictive value and wherein the sensitivity of said method is at least 90%, specificity is at least 60%, negative predictive value in a population having a cat allergy prevalence of at least 75% and positive predictive value in a population having a cat allergy prevalence of at least 80%.

In certain non-limiting embodiments, there is provided a method of diagnosing cat allergy in an individual by skin prick method using the kit, wherein clinical parameters are selected from the group consisting of sensitivity, specificity, negative predictive value, and positive predictive value and wherein the sensitivity, specificity, negative predictive value and positive predictive value is of about 100%.

According to one embodiment of the processes described herein, the allergenic intermediate is reconstituted after lyophilization. For example, lyophilized material may be combined with reconstitution fluid at a ratio to target a particular concentration of allergenic extract (e.g., 30,000 BAU/mL). A suitable reconstitution fluid may include, for example and without limitation, a glycerol-cocas fluid (0.5% sodium chloride, 0.275% sodium bicarbonate, and 52.5% glycerin, in water for injection).

In one embodiment, the present invention provides a kit for compounding allergenic intermediate comprising: a) a first container comprising a lyophilized allergenic intermediate, b) a second container comprising a liquid diluent, and c) optionally instructions for preparation and use, wherein the content of the first and second containers can be combined to achieve a concentration of allergenic extract in the range of at least about 10,000 to about 120,000 BAU/mL.

In one embodiment, the present invention provides a kit for compounding an allergenic intermediate comprising: a) a first container comprising a lyophilized allergenic intermediate, b) a second container comprising a liquid diluent, and c) optionally instructions for preparation and use, wherein the content of the first and second containers can be combined to achieve 30,000 BAU/mL concentration of allergenic extract.

In one embodiment, the second container of the kit comprises a liquid diluent comprising glycerol-cocas fluid. In a further embodiment, the glycerol-cocas fluid comprises 0.5% sodium chloride, 0.275% sodium bicarbonate, and 52.5% glycerin in water for injection.

In one embodiment, the present invention provides a kit for compounding allergenic intermediate comprising: a) first container comprising a lyophilized allergenic intermediate, b) second container comprising a liquid diluent comprising glycerol-cocas fluid (0.5% sodium chloride, 0.275% sodium bicarbonate, and 52.5% glycerin in water for injection), and c) optionally instructions for preparation and use, wherein the content of the first and second containers can be combined to achieve a concentration of allergenic extract in the range of at least about 10,000 to about 120,000 BAU/mL.

In one embodiment, the present invention provides a kit for compounding allergenic intermediate of the present invention comprising: a) a first container comprising a lyophilized allergenic intermediate, b) a second container comprising a liquid diluent comprising glycerol-cocas fluid (0.5% sodium chloride, 0.275% sodium bicarbonate, and 52.5% glycerin in water for injection), and c) optionally instructions for preparation and use, wherein the content of the first and second containers can be combined to achieve 30,000 BAU/mL concentration of allergenic extract.

In one embodiment, the present invention provides a kit for compounding allergenic intermediate, wherein the allergenic intermediate and the liquid diluent each is pre-measured into a respective unit of amount.

The terms used in the present invention such as ‘a kit’ or a compounding kit′ and ‘a kit for compounding’, are interchangeable. The kit as per the present invention may comprise one, two or more containers, wherein the first and second containers have pre-measured amounts of components in a respective unit of amount, such that the powder and liquid of the first and second containers can be combined in either the first or second container to achieve a desired concentration of allergenic extract. As used herein, the term “container”, “containers”, “pack”, or “packs” include kit, compounding kit for reconstitution, bottles, vials, ampoules, jars, pouches, sachets, blisters, syringes, prefilled syringes, bags, injectors and the like. The instructions for preparation and use can be in the form of a printed leaflet. The kit as per the present invention may also comprise suitable devices for use of allergenic extract as per the present invention such as ComforTen Device or any other devices known in the art.

The highly concentrated allergen extract of the present invention is expected to exhibit desired clinical advantages. Allergic patients, with a diagnosis of allergy, may be administered one, two, or three single doses and/or multiple doses of an allergenic extract described herein. In one embodiment, the dose to be administered to an allergic patient is a single dose. In administering a dose or dosages of the allergenic extract described herein, the patient will be observed for a specific period of time for therapeutic response and for any symptoms. Side effects and tolerability of test samples can be recorded after each dose to the patient. Thus, the compositions tested are expected to be feasible for clinical use in allergen immunotherapy.

Having described the invention with reference to certain preferred embodiments, other embodiments will become apparent to one skilled in the art from consideration of the specification. The invention is further defined by reference to the following examples describing in detail the preparation of Cat Hair-Dander Allergen. It will be apparent to those skilled in the art that many modifications, both to materials and methods, may be practiced without departing from the scope of the invention.

The following examples are provided to illustrate embodiments of the disclosure but they are by no means intended to limit its scope. The scope may include, but is not limited to, naturally occurring allergens include animal hair and dandruff allergens (from e.g., dog, cat, horse, rabbit, rat, mouse, etc.), pollen allergens (e.g., tree, herb, weed, and grass pollen allergens), insect allergens (e.g., inhalant, saliva, and venom allergens, such as from mite, cockroach, midge, or hymenoptera venom), and food allergens and/or any other as known in the art.

### EXAMPLES

### Example 1—Cat Hair-Dander Manufacture; 30,000 BAU/mL Extract

Step 1—Extraction of Cat Hair-Dander (Extraction Mixture)

Cat hair-dander source material was combined with extraction fluid (0.1% sodium chloride, 0.2% sodium bicarbonate, 0.5% liquefied phenol, in water for injection; pH 8.0-9.0) at a ratio of 1 g:6.4 mL. Nine containers, each containing 10,000 g of cat hair-dander source material and 64,000 mL of extraction fluid were prepared. Extraction of cat hair-dander source material continued for 5 days at 1° C.-5° C. to form an extraction mixture.

Step 2—Initial Clarification of Cat Hair-Dander Extract (Clarified Extract)

To remove gross solids (i.e., hair) the extraction mixture of Step 1 was centrifuged in an industrial-sized perforated basket-style centrifuge. The liquid portion was collected and the solids were disposed of. The recovered extract was then further clarified using Whatman filter paper, with the help of a filter-aid. The clarified liquid was collected into a holding tank.

Step 3—Pervaporation of Cat Hair-Dander Extract (Concentrated Extract)

The clarified extract of Step 2 was then concentrated to a 10% target volume by pervaporation in dialysis casing (molecular weight cut-off 12-16 kDa) at room temperature (20° C.-25° C.). The pH of the concentrated extract was adjusted to a pH of 4.8 using 5 N HCl.

Step 4—Acetone Precipitation of Concentrated Cat Hair-Dander Extract (Precipitated Solids)

From the concentrated extract of Step 3, the proteins and allergens were precipitated out of solution using an acetone treatment. Chilled acetone was added to the concentrated extract at a ratio of 4 parts acetone to 1 part extract ratio. Following mixing for approximately 5 minutes, the mixture was allowed to settle for 16 to 17 hours. The acetone was then poured off to give precipitated solids.

Step 5—Freeze Drying of Acetone Precipitated Cat Hair-Dander Material (Cat Hair Dander Intermediate Raw Material)

The recovered precipitate solids of Step 4 were aerated, uncovered in an exhaust hood, for 120 hours. Following aeration, the precipitate was reconstituted in 7,200 mL of cold Water For Injection (WFI) and then transferred into trays. The material was frozen and then freeze-dried to form a brown, flaky raw material as an intermediate raw material.

Step 6—Extraction of Cat Hair-Dander Intermediate Raw Material (Glycerinated Extract)

The cat hair-dander raw material intermediate of Step 5 was combined with extraction fluid (0.5% sodium chloride, 0.275% sodium bicarbonate, and 52.5% glycerin, in water for injection) at a ratio to target a 30,000 BAU/mL extract. The formulation was calculated based on the Fel d 1 content of the raw material batches used. For a one liter batch, 22.6 grams of raw material was added to one liter of extraction fluid. The mixture was continuously mixed at room temperature (20° C.-25° C.) for 15 hours, 18 minutes to extract proteins and allergens to give a glycerinated extract.

Step 7—Clarification of Glycerinated Cat Hair-Dander Extract

A filter-aid was added to the recovered extract, followed by filtration at room temperature using Whatman filter paper. Room temperature filtration continued using a 3M/CUNO Zeta Plus 50 CP depth filter; and finally a using a 500 cm′ Pall Supor (PES), 0.8/0.2 μm capsule filter. The clarified liquid was collected into a holding tank and stored at 2° C.-8° C. pending sterile filtration.

The extract was sterile filtered at room temperature (20° C.-25° C.) using Pall Supor (PES), 0.8/0.2 μm capsule filters. The sterile filtered extract was stored in bulk containers at 2° C.-8° C. pending the filling of final container vials to form Cat Hair Allergen Extracts.

### Example 2

A clinical trial in 32 subjects was accomplished to confirm the viability and efficacy and/or diagnostic performance of the present invention.

### Experimental Design:

A single-center, phase I cohort study was performed to evaluate the relative capability of different cat hair allergen extracts to diagnose cat allergy by skin prick testing.

### Materials and Methods:

Allergic patients, with a diagnosis of cat hair allergy and non-allergic subjects were administered doses at 10,000, 30,000, and 90,000 BAU/mL of a Cat Hair Allergen Extracts, in a randomized, single-center, Phase I Cohort study. The subjects were evaluated using skin prick testing.
The allergens that were used in the clinical trials for diagnostic skin prick test were Cat hair allergen extract 10,000 BAU/mL; 30,000 BAU/mL; and 90,000 BAU/mL (Test).
Allergenic Extract, Standardized Cat Pelt AP, 10,000 BAU/mL (by Jubilant HollisterStier LLC) was used as a reference product (Reference) used for diagnostic nasal provocation test.
The clinical trial had the following objectives and parameters:
1. The primary objective was to determine the sensitivity, specificity, and positive and negative predictive values of the Cat hair allergen extract 30,000 BAU/mL (Test) by Skin Prick Test versus Nasal Provocation Test (Reference).
2. Another primary objective of the study was to determine the safety of the Cat hair allergen extract 30,000 BAU/mL (Test) by Skin Prick Test.
3. The secondary objective was to determine the sensitivity, specificity, and positive and negative predictive values of the Cat hair allergen extract 10,000 BAU/mL and BAU/mL (Test) by Skin Prick Test versus Nasal Provocation Test (Reference).
4. Another secondary objective of the study was to assess the dose-response of Cat hair allergen extract at 10,000 BAU per mL, 30,000 BAU per mL, and 90,000 BAU per mL (Test) using the Skin Prick Test erythema/wheal diameter.
5. The evaluation was performed in both clinically allergic and non-allergic subjects.

### Results:

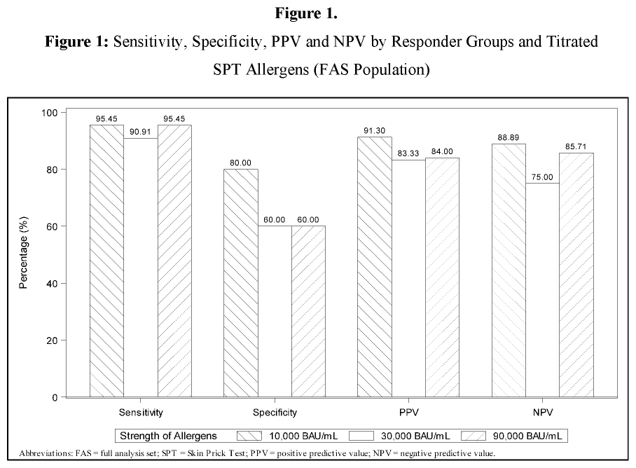

1. Sensitivity, Specificity, Positive Predictive Value (PPV), and Negative Predictive Value (NPV):
Sensitivity, Specificity, Positive Predictive Value (PPV), and Negative Predictive Value (NPV) the Cat hair allergen extracts, 10,000 BAU/mL, 30,000 BAU/mL, BAU/mL, by Skin Prick Test (SPT) versus Nasal Provocation Test (NPT) is represented in FIG. 1 for all 32 subjects.
For 10,000 BAU/mL, PPV was 91.30% and NPV was 88.89% by Skin Prick Test (SPT). For 30,000 BAU/mL, PPV was 83.33% and NPV was 75.00% by Skin Prick Test (SPT). For 90,000 BAU/mL, PPV was 84.00% and NPV was 85.71% by Skin Prick Test (SPT).
For 10,000 BAU/mL, sensitivity was 95.45% and specificity was 80.00% by Skin Prick Test (SPT). For 30,000 BAU/mL, sensitivity was 90.91% and specificity was 60.00% by Skin Prick Test (SPT). For 90,000 BAU/mL sensitivity was 95.45% and specificity was 60.00% by Skin Prick Test (SPT).

2. Safety of Cat Hair Allergen Extracts:
The safety of Cat Hair Allergen Extracts 30,000 BAU/mL by Skin Prick Test (SPT) was determined for all of the 32 subjects enrolled. There were no suspected unexpected serious adverse drug reactions observed during the study.

3. Dose-Response of Cat Hair Allergen Extract:
The dose-response of Cat hair allergen extract at 10,000 BAU per mL, 30,000 BAU per mL, and 90,000 BAU per mL was assessed using the SPT wheal and erythema diameters. The results are represented in FIGS. 2 and 3.

What is claimed:

1. A process for preparing a highly concentrated allergenic intermediate from a dilute allergen extract, said process comprising:
providing a crude extract comprising biological material from an allergenic source, wherein the biological material comprises a solid phase portion and a liquid phase portion;
concentrating the crude extract using pervaporation;
adjusting the pH of the concentrated crude extract to 4.0-5.0;
treating the concentrated extract with a ketonic solvent to form a precipitate; and
recovering the precipitate by filtration to obtain a highly concentrated allergenic intermediate.

2. The process according to claim 1, wherein said providing a crude extract comprises:
contacting an allergenic source material with an extraction fluid;
extracting biological material from the allergenic source into the extraction fluid, wherein said extracting is carried out with no adjustment to pH; and
separating the biological material into a solid phase portion and a liquid phase portion to obtain a crude extract.

3. The process according to claim 2, wherein the allergenic source material is selected from house dust mite allergen, insect allergen, venom allergen, animal hair and dander allergen, food allergen, and combinations thereof.

4. The process according to claim 2, wherein said allergenic source material is contacted with the extraction fluid at a concentration ratio of 1:4 to 1:8.

5. The process according to claim 2, wherein said extracting is carried out for 2-6 days.

6. The process according to claim 2, wherein said extracting is carried out at a temperature of −5° C. to 30° C.

7. The process according to claim 2, wherein said allergenic source material comprises animal hair and dander allergen.

8. The process according to claim 7, wherein said allergenic source is contacted with said extraction fluid at a concentration ratio of 1:6-7.

9. The process according to claim 7, wherein said extracting is carried out for 3-5 days.

10. The process according to claim 2, wherein said extracting is carried out at a temperature of 0° C.-5° C.

11. The process according to claim 2, wherein said separating is carried out using filtration, centrifugation, or a combination thereof.

12. The process according to claim 1, wherein said concentrating is carried out in the absence of a conductivity test.

13. The process according to claim 1, wherein said concentrating is carried out to a concentration equal to or less than 20% of original volume.

14. The process according to claim 1 further comprising:
repeating said treating.

15. The process according to claim 1 further comprising:
subjecting the precipitate to lyophilization or vacuum drying after said recovering.

16. The process according to claim 15 further comprising:
subjecting the lyophilized or vacuum dried precipitate to reconstitution to form a highly concentrated extract.

17. A kit for compounding allergenic intermediate comprising:
a) a first container comprising a lyophilized allergenic intermediate,
b) a second container comprising a liquid diluent, and
c) optionally instructions for preparation and use,
wherein the content of first and second containers can be combined to achieve BAU/mL concentration of allergenic extract.

18. The kit according to claim 17, wherein the second container comprises a liquid diluent comprising glycerol-cocas fluid.

19. The kit according to claim 18, wherein the glycerol-cocas fluid comprises sodium chloride, 0.275% sodium bicarbonate, and 52.5% glycerin in water for injection.

20. A method of diagnosing cat allergy in an individual by skin prick method using the kit according to claim 17, wherein clinical parameters are selected from the group consisting of sensitivity, specificity, negative predictive value, and positive predictive value and wherein the sensitivity of said method is at least 90%, specificity is at least 60%, negative predictive value in a population having a cat allergy prevalence of at least 75% and positive predictive value in a population having a cat allergy prevalence of at least 80%.
